(19) United States
(12) Patent Application Publication
(10) Pub. No.: US 2026/0345467 A1
(43) Pub. Date: Nov. 5, 2026

(54) OIL-BASED LIP COSMETIC

(71) Applicant: SHISEIDO COMPANY, LTD., Chuo-ku, Tokyo (JP)

(72) Inventors: Keisuke HAYASHIDA, Tokyo (JP); Kyohei KOSAKA, Tokyo (JP); Mizuki YAMAMOTO, Tokyo (JP); Kiriko CHIBA, Tokyo (JP)

(73) Assignee: SHISEIDO COMPANY, LTD., Chuo-ku, Tokyo (JP)

(21) Appl. No.: 19/115,499

(22) PCT Filed: Oct. 12, 2023
(86) PCT No.: PCT/JP2023/037033

§ 371 Date: Mar. 26, 2025

(30) Foreign Application Priority Data
Oct. 26, 2022 (JP) 2022-171207
Mar. 24, 2023 (JP) 2023-047686

(51) Int. Cl. A61K 8/891; A61K 8/19; A61K 8/893; A61K 8/894; A61Q 1/04

(52) U.S. Cl. CPC A61K 8/891; A61K 8/19; A61K 8/893; A61K 8/894; A61Q 1/04; A61K 2800/412; A61K 2800/43; A61K 2800/651; A61K 2800/652

(57) ABSTRACT

An objective of the invention is to provide an oil-based lip cosmetic that is resistant to color transfer to masks, cups, etc. (secondary adhesion-less effects) and that has good color retention. The oil-based lip cosmetic according to the present invention is characterized by containing (A) a phenyl-modified silicone; (B) 1.5% to 15% by mass, with respect to the overall amount of the cosmetic, of a silicone-based surfactant; and (C) 5% to 35% by mass, with respect to the overall amount of the cosmetic, of a powder component selected from among (i) extender pigments and (ii) polymer powders.

### TECHNICAL FIELD

The present invention relates to an oil-based lip cosmetic. More specifically, the present invention relates to an oil-based lip cosmetic that is resistant to color transfer to masks, cups, etc. (secondary adhesion-less effects) and that has good color retention.

### BACKGROUND ART

Even among lip cosmetics, oil-based lip cosmetics such as lipsticks can be made compact and are easily portable, and have the convenience of being able to be directly applied to the lips without using the fingers or tools.

In oil-based lip cosmetics, the feature of blending large amounts of high-viscosity hydrocarbon oils such as hydrogenated polyisobutene or pentaerythrityl tetraisostearate, which adhere to the lips, is widely practiced. However, the feature of blending large amounts of high-viscosity hydrocarbon oils has the drawback that, in addition to becoming difficult to spread at the time of use, the finish being sticky, etc., a coating film can be transferred, oil components and all, to portions contacted by the lips. As a result thereof, there was a problem in that color transfer occurs over time and the color retention is poor.

Therefore, oil-based lip cosmetics that are resistant to secondary adhesion, having so-called secondary adhesion-less effects, have been developed.

For example, Patent Document 1 proposes a lip cosmetic comprising a phenyl-modified silicone, a specific liquid multi-branched isostearic acid ester compound, a specific alkyl glyceryl ether-modified silicone, an oil having a viscosity of 100 mPa·s or lower at 30 °C and having at least one hydroxyl group in each molecule, and a wax. This lip cosmetic provides secondary adhesion-less effects by the phenyl-modified silicone, which was held in the wax structure during application, being exposed to the coating film surface and forming a film.

However, this lip cosmetic has the problem that the specific alkyl glyceryl ether-modified silicone is essential, thus limiting the formulation. In particular, when making combined use of powder components, aggregation of the powders tends to occur. Therefore, large amounts of powder components cannot be blended. Furthermore, the secondary adhesion-less effects could not yet be considered to have attained a fully satisfactory level.

### RELATED ART

### Patent Documents

Patent Document 1: JP 2014-19663 A

### SUMMARY OF THE INVENTION

### Problem to be Solved by the Invention

The present invention was made in consideration of the above-mentioned circumstances, and an objective thereof is to provide an oil-based lip cosmetic that is resistant to color transfer to masks, cups, etc. (secondary adhesion-less effects) and that has good color retention.

### Means for Solving the Problem

The present inventors carried out diligent investigations towards solving the above-mentioned problem, as a result of which they discovered that the above-mentioned problem can be solved by blending, in prescribed amounts, (A) a phenyl-modified silicone, (B) a silicone-based surfactant and (C) a prescribed powder component, thereby completing the present invention.

That is, the gist of the present invention is an oil-based lip cosmetic containing:
(A) a phenyl-modified silicone;
(B) 1.5% to 15% by mass, with respect to the overall amount of the cosmetic, of a silicone-based surfactant; and
(C) 5% to 35% by mass, with respect to the overall amount of the cosmetic, of a powder component selected from among (i) extender pigments and (ii) polymer powders.

### Effects of the Invention

The oil-based lip cosmetic according to the present invention can significantly suppress color transfer to masks, cups, etc. (secondary adhesion-less effects) due to the above-mentioned features. Additionally, since the coating film is resistant to being removed from the lips, the present invention also has excellent retention of makeup effects, i.e., color retention.

### MODES FOR CARRYING OUT THE INVENTION

The oil-based lip cosmetic of the present invention is characterized by comprising (A) a phenyl-modified silicone, (B) a silicone-based surfactant, and (C) a powder component. Hereinafter, the respective components constituting the oil-based lip cosmetic of the present invention will be explained in detail.

### <(A) Phenyl-Modified Silicone>

The (A) phenyl-modified silicone is a silicone that has a dimethylsiloxane backbone, with at least some of the methyl groups substituted by phenyl groups, which can be used without any particular limitations as long as it can be used in a cosmetic. Specific examples include diphenyl dimethicone, diphenylsiloxy phenyl trimethicone, trimethyl pentaphenyl trisiloxane, polysilicone-32, phenyl trimethicone and methylphenyl polysiloxane. Among these, diphenyl dimethicone, diphenylsiloxy phenyl trimethicone, trimethyl pentaphenyl trisiloxane and polysilicone-32 are particularly preferable, and diphenyl dimethicone is particularly preferable from the aspect of compatibility with the (B) silicone-based surfactant.

As commercial products, examples include DOWSIL SH-556 Fluid, FZ-209 (the above from Dow Toray Co., Ltd.), KF-50-100cs, KF-50-1000cs, KF-53, KF-54, KF-54HV, KF-56A, KSG-18A (the above from Shin-Etsu Chemical Co., Ltd.), Silshine MP Fluid (Momentive Performance Materials Japan, LLC), etc.

The (A) phenyl-modified silicone can be used as a single type or as a combination of two or more types. The blended amount of the (A) phenyl-modified silicone used in the present invention is preferably 20% by mass or more, more preferably 30% by mass or more, and 75% by mass or less, more preferably 70% by mass or less, with respect to the overall amount of the oil-based lip cosmetic. That is, examples of the blended amount range are 20% to 75% by mass, 30% to 70% by mass, etc. Within these ranges, the secondary adhesion-less effects and good color retention are adequately achieved, and in the case in which the cosmetic is solid, a stick shape can be stably retained and a good sense of glossiness is also obtained.

### <(B) Silicone-Based Surfactant>

The (B) silicone-based surfactant is a surfactant having a structure in which hydrophilic substituent groups are introduced to a portion of a silicone having siloxane bonds in a main backbone. Preferable modes of the (B) silicone-based surfactant that can be used in the present invention include (i) polyoxyalkylene-modified silicones, (ii) polyoxyalkylene/alkyl-comodified silicones, (iii) polyglycerin-modified silicones, and (iv) partially-crosslinked-type modified silicone gels. Among these, PEG-10 dimethicone or PEG-12 dimethicone are particularly preferable from the aspect of compatibility with the (A) phenyl-modified silicone.

Commercial products that are (B) silicone-based surfactants include, for example: as the (i) polyoxyalkylene-modified silicones, Silicone SC9450N (PEG-10 dimethicone, Shin-Etsu Chemical Co., Ltd.), Silicone KF-6017P (PEG-10 dimethicone, Shin-Etsu Chemical Co., Ltd.), Silicone SH3773M (PEG-12 dimethicone, Shin-Etsu Chemical Co., Ltd.), SILWET 10-E (PEG-10 methyl ether dimethicone, Shin-Etsu Chemical Co., Ltd.), Silicone KF6018 (PEG-11 methyl ether dimethicone, Shin-Etsu Chemical Co., Ltd.) and Silicone SC0938B (PEG-9 polydimethylsiloxyethyl dimethicone, Shin-Etsu Chemical Co., Ltd.); as the (ii) polyoxyalkylene/alkyl-comodified silicones, Silicone SC0928SL (lauryl PEG-9 polydimethylsiloxyethyl dimethicone, Shin-Etsu Chemical Co., Ltd.) and ABIL EM90 (cetyl PEG/PPG-10/1 dimethicone, Evonik Goldschmidt Corp.); as the (iii) polyglycerin-modified silicones, Silicone KF6109 (bis-butyl dimethicone polyglyceryl-3, Shin-Etsu Chemical Co., Ltd.); and as the (iv) partially crosslinked-type modified silicone gels, KSG-210 ((dimethicone/(PEG-10/15)) crosspolymer, Shin-Etsu Chemical Co., Ltd.), KSG-710 ((dimethicone/polyglycerin-3) crosspolymer, Shin-Etsu Chemical Co., Ltd.) and KSG-360Z ((PEG-15/lauryl polydimethylsiloxyethyl dimethicone) crosspolymer, Shin-Etsu Chemical Co., Ltd.).

Although the alkyl glyceryl ether-modified silicones blended in Patent Document 1 are also classified as silicone-based surfactants, they tend to cause powder aggregation so that a large amount of the (C) powder component cannot be blended. Thus, they are not suitable for use in the present invention.

The (B) silicone-based surfactant can be used as a single type or as a combination of two or more types. The blended amount of the (B) silicone-based surfactant used in the present invention is preferably 1.5% by mass or more, more preferably 3% by mass or more, and 15% by mass or less, more preferably 10% by mass or less, and even more preferably 8% by mass or less, with respect to the overall amount of the oil-based lip cosmetic. Therefore, the blended amount of the (B) silicone-based surfactant may be 1.5% to 15% by weight, etc. with respect to the overall amount of the cosmetic.

If the blended amount of the (B) silicone-based surfactant is within the above-mentioned range, secondary adhesion-less effects and good color retention are adequately achieved, and in the case in which the cosmetic is solid, a stick shape can be stably retained.

### <(C) Powder Component>

As the (C) powder component blended into the present invention, among powder components that are used in cosmetics, powders selected from among (i) extender pigments and (ii) polymer powders can be used.

The average particle size of the (C) powder component is preferably 2 μm or larger, and preferably 20 μm or smaller. Therefore, the average particle size of the (C) powder component may be, for example, 2 to 20 μm, etc. If the average particle size is smaller than 2 μm, there are cases in which it becomes difficult to uniformly disperse the powder component in the oil-based lip cosmetic, and if the average particle size is larger than 20 μm, there are cases in which the feeling in use becomes poor.

The “average particle size” in the present specification is a value measured by a laser scattering and diffraction method. This value may, for example, be measured by using a Microtrac HRA (manufactured by Microtrac Corp.), etc.

As the (i) extender pigments, clay minerals or synthetic inorganic powders can be used. For example, mica, silica, alumina, barium sulfate, titanium oxide, synthetic phlogopite, boron nitride, and complex powders thereof are preferable.

As the (ii) polymer powders, synthetic or natural polymer powders can be used. For example, (vinyl dimethicone/methicone silsesquioxane) crosspolymer, (diphenyl dimethicone/vinyl diphenyl dimethicone/silsesquioxane) crosspolymer, cellulose and (HDI/trimethylol hexyl lactone) crosspolymer are preferable.

The (C) powder components may have the surfaces thereof hydrophobized as needed. The hydrophobization treatment may include, for example, silicone treatments (treatments with silicone oils such as methylhydrogen polysiloxane, dimethyl polysiloxane and methyphenyl polysiloxane; with alkyl silanes such as methyl trimethoxysilane, ethyl trimethoxysilxane, hexyl trimethoxysilanea and octyl trimethoxysilane; and with fluoroalkyl silanes such as trifluoromethylethyl trimethoxysilane and heptadecafluorodecyl trimethoxysilane, etc.), fatty acid treatments (treatments with palmitic acid, isostearic acid, stearic acid, lauric acid, myristic acid, behenic acid, oleic acid, rosinic acid, 12-hydroxystearic acid, etc.), fatty acid soap treatments (treatments with aluminum stearate, calcium stearate, etc.) and fatty acid ester treatments (treatments with dextrin fatty acid ester, cholesterol fatty acid ester, sucrose fatty acid ester, starch fatty acid ester, etc.). These hydrophobization treatments can be performed in accordance with conventional methods.

As the (C) powder component, one or both of an (i) extender pigment and a (ii) polymer powder may be used, and furthermore, each of the (i) extender pigment and the (ii) polymer powder can be used as a single type alone or as a combination of two or more types. The blended amount of the (C) powder component used in the present invention is preferably 5% by mass or more, and preferably 35% by mass or less with respect to the overall amount of the oil-based lip cosmetic. Therefore, the blended amount of the (C) powder component can be 5% to 35% by mass, etc. with respect to the overall amount of the cosmetic.

If the blended amount of the (C) powder component is within the above-mentioned range, the strength of the coating film increases, secondary adhesion-less effects and good color retention are adequately achieved, and in the case in which the cosmetic is solid, excellent stick shape retention is also obtained.

### <Optional Blended Components>

Aside from the components (A) to (C) above, oil-based gelling agents, oils, colorants, water, polymer compounds, humectants, fragrances, antioxidants, preservatives, beautifying components, ultraviolet absorbents, etc. that are used in normal oil-based lip cosmetics can be blended, as appropriate, in the oil-based lip cosmetic of the present invention, within a range not compromising the effects of the present invention.

Examples of oil-based gelling agents include dextrin fatty acid esters, glycerin fatty acid esters, organically modified clay minerals, waxes, etc. Dextrin fatty acid esters include, for example, dextrin myristate, dextrin palmitate, dextrin (palmitate/2-ethylhexanoate), etc. Glycerin fatty acid esters include, for example, glyceryl behenate, glyceryl octastearate, glyceryl eicosanoate, etc. Organically modified clay minerals include, for example, dimethyl distearyl ammonium hectorite (disteardimonium hectorite), dimethyl alkyl ammonium hectorite, benzyl dimethyl stearyl ammonium hectorite, distearyl dimethyl ammonium chloride-treated aluminum-magnesium silicate, etc. Waxes include, for example, carnauba wax, candelilla wax, polyethylene wax, paraffin wax, beeswax, ceresin, microcrystalline wax, solid paraffin, Japan wax, etc. Among these, a hydrocarbon wax such as polyethylene wax, paraffin wax or microcrystalline wax is particularly preferable, since including these improves the solidification power (or moldability) when forming the oil-based lip cosmetic in the shape of a stick.

Examples of oils include ester oils such as diisostearyl malate, sucrose tetraisostearate, triisostearin, diglyceryl triisostearate (polyglyceryl-2 triisostearate), di(phytosteryl/octyldodecyl) lauroyl glutamate, diisopropyl sebacate, triethylhexanoin, isopropyl myristate and diethylhexyl succinate; hydrocarbon oils such as mineral oils, hydrogenated polyisobutene (hydrogenated isopolybutene) and hydrogenated polydecene; and silicone oils such as dimethicone; etc. However, the oils are not limited to the above.

As the colorant, one that is normally blended into oil-based lip cosmetics, such as a pigment or a dye, may be used. Examples of the colorant include inorganic white pigments (titanium dioxide and zinc oxide); inorganic red pigments (red iron oxide (Bengal red) and iron titanate); inorganic brown pigments (γ-iron oxide); inorganic yellow pigments (yellow iron oxide and ocher); inorganic black pigments (black iron oxide, carbon and low-order titanium oxide); inorganic violet pigments (mango violet and cobalt violet); inorganic green pigments (chromium oxide, chromium hydroxide and cobalt titanate); inorganic blue pigments (ultramarine blue and Prussian blue); metal powder pigments (aluminum powder and copper powder); organic pigments (Red No. 202, Red No. 205, Red No. 220, Red No. 228, Red No. 405, Orange No. 203, Orange No. 204, Yellow No. 205, Yellow No. 401 and Blue No. 404); organic pigments that are zirconium, barium or aluminum lakes (Red No. 3, Red No. 104, Red No. 227, Red No. 401, Orange No. 205, Yellow No. 4, Yellow No. 202, Green No. 3 and Blue No. 1); natural pigments (chlorophyll, carotenoids (β-carotene), carthamin, cochineal, chalcone, curcumin, betanin, flavonol, flavone, anthocyanidin, anthraquinone and naphthoquinone); pearlescent pigments (titanium oxide-coated mica, titanium oxide-coated bismuth oxychloride, titanium oxide-coated talc, colored titanium oxide-coated mica, bismuth oxychloride and argentine); etc.

Although the colorants such as the above-mentioned inorganic colored pigments and pearlescent pigments are in powdered form, sufficient secondary adhesion-less effects cannot be obtained even when these are blended instead of the (C) powder component. Additionally, since there are cases in which the secondary adhesion-less effects are hindered when these powder colorants are blended in large amounts, these should not be blended, or even if blended, should be kept at ½ or less, more preferably ⅓ or less, and even more preferably ⅕ or less of the (C) powder component.

Water may also be blended into the oil-based lip cosmetic of the present invention. However, if the water content is high, the stability becomes poor. Thus, it should be held to 2% by mass or less with respect to the overall amount of the oil-based lip cosmetic.

The specific format of the oil-based lip cosmetic of the present invention may be of any type that is directly applied to the lips, and may be in stick form or paste form, with the stick form being particularly preferable.

### EXAMPLES

Though the present invention will be explained in further detail by providing examples below, the present invention is not limited in any way by these examples. Where not specially noted, the blended amounts are indicated in percentage by mass relative to the systems in which the relevant components are blended. Before specifically explaining the respective examples, the evaluation methods that were employed will be explained.

### <Secondary Adhesion-Less Effects>

Respective samples of examples and comparative examples were evaluated regarding the secondary adhesion-less effects by using the tapping test below.

### [Tapping Test]

The sample was applied in an amount of 2.5 mg/cm² to white synthetic leather (4 cm²), the synthetic leather was folded with the application surface on the inside, pinched between the fingers and squeezed five times, and placed at rest for five minutes. Next, the synthetic leather was attached to a tapping tester with the application surface on the outside, and tapping was performed twice (N=3) with respect to filter paper. The secondary adhesion-less effects were assessed by visual inspection from the degree of adhesion of the sample to the filter paper after tapping.

### [Evaluation Criteria]

A: Almost no adhesion observed
B: Slight adhesion observed, yet of a fully acceptable level
C: Adhesion of an unacceptable level observed

### Examples 1 to 17 and Comparative Examples 1 and 2

Oil-based lip cosmetics having the compositions described in Table 1A and Table 1B below were prepared and evaluated regarding secondary adhesion-less effects.

TABLE 1A

| | Ex. | Ex. | Ex. | Ex. | Ex. | Ex. | Ex. | Ex. | Ex. | Ex. |
|---|---|---|---|---|---|---|---|---|---|---|
| | 1 | 2 | 3 | 4 | 5 | 6 | 7 | 8 | 9 | 10 |
| Polyethylene/microcrystalline | 6 | 6 | 6 | 6 | 6 | 6 | 6 | 6 | 6 | 6 |
| wax | | | | | | | | | | |
| Polysilicone-32 | 25 | 20 | 15 | 10 | 5 | — | 25 | 25 | 25 | 25 |
| Diphenylsiloxy phenyl | 29.98 | 34.98 | 39.98 | 44.98 | 49.98 | 54.98 | 31.98 | 33.98 | 35.98 | 34.98 |
| trimethicone | | | | | | | | | | |
| Diphenyl dimethicone | — | — | — | — | — | — | — | — | — | — |
| PEG-10 dimethicone | 8 | 8 | 8 | 8 | 8 | 8 | 6 | 4 | 2 | 8 |
| Mica⁽¹⁾ | 15 | 15 | 15 | 15 | 15 | 15 | 15 | 15 | 15 | 10 |
| Polyglyceryl-2 triisostearate | 10 | 10 | 10 | 10 | 10 | 10 | 10 | 10 | 10 | 10 |
| Hydrogenated polyisobutene | 5 | 5 | 5 | 5 | 5 | 5 | 5 | 5 | 5 | 5 |
| Dimethicone | 0.02 | 0.02 | 0.02 | 0.02 | 0.02 | 0.02 | 0.02 | 0.02 | 0.02 | 0.02 |
| Red No. 202 | 1 | 1 | 1 | 1 | 1 | 1 | 1 | 1 | 1 | 1 |
| Total | 100 | 100 | 100 | 100 | 100 | 100 | 100 | 100 | 100 | 100 |
| Secondary adhesion-less effects | A | B | B | B | B | B | A | A | B | A |

⁽¹⁾Average particle size 5.2 μm

TABLE 1B

| | Ex. | Ex. | Ex. | Ex. | Ex. | Ex. | Ex. | Comp. | Comp. |
|---|---|---|---|---|---|---|---|---|---|
| | 11 | 12 | 13 | 14 | 15 | 16 | 17 | Ex. 1 | Ex. 2 |
| Polyethylene/microcrystalline | 6 | 6 | 6 | 6 | 6 | 6 | 6 | 6 | 6 |
| wax | | | | | | | | | |
| Polysilicone-32 | 25 | — | — | — | — | — | — | 25 | 25 |
| Diphenylsiloxy phenyl | 39.98 | 29.98 | 34.98 | 39.98 | 44.98 | 49.98 | 54.98 | 37.98 | 44.98 |
| trimethicone | | | | | | | | | |
| Diphenyl dimethicone | — | 25 | 20 | 15 | 10 | 5 | — | — | — |
| PEG-10 dimethicone | 8 | 8 | 8 | 8 | 8 | 8 | 8 | — | 8 |
| Mica⁽¹⁾ | 5 | 15 | 15 | 15 | 15 | 15 | 15 | 15 | — |
| Polyglyceryl-2 triisostearate | 10 | 10 | 10 | 10 | 10 | 10 | 10 | 10 | 10 |
| Hydrogenated polyisobutene | 5 | 5 | 5 | 5 | 5 | 5 | 5 | 5 | 5 |
| Dimethicone | 0.02 | 0.02 | 0.02 | 0.02 | 0.02 | 0.02 | 0.02 | 0.02 | 0.02 |
| Red No. 202 | 1 | 1 | 1 | 1 | 1 | 1 | 1 | 1 | 1 |
| Total | 100 | 100 | 100 | 100 | 100 | 100 | 100 | 100 | 100 |
| Secondary adhesion-less effects | A | A | B | B | B | B | B | C | C |

As indicated in Table 1A and Table 1B above, the oil-based lip cosmetics containing (A) a phenyl-modified silicone (polysilicone-32, diphenylsiloxy phenyl trimethicone and diphenyl dimethicone), (B) a silicone-based surfactant (PEG-10 dimethicone) and (C) a powdered component (mica) all exhibited adequate secondary adhesion-less effects (Examples 1-17). A tendency to have excellent secondary adhesion-less effects was observed particularly in the case in which large amounts of polysilicone-32 and diphenyl dimethicone were blended as the (A) phenyl-modified silicone (Examples 1, 7, 8, 10, 11 and 12).

On the other hand, adequate secondary adhesion-less effects were not obtained in the cases in which (B) a silicone-based surfactant or (C) a powdered component were not contained (Comparative Examples 1 and 2).

### Examples 18 to 39

Oil-based lip cosmetics having the compositions described in Table 2A and Table 2B below were prepared and evaluated regarding secondary adhesion-less effects.

TABLE 2A

| | Ex. | Ex. | Ex. | Ex. | Ex. | Ex. | Ex. | Ex. | Ex. | Ex. | Ex. |
|---|---|---|---|---|---|---|---|---|---|---|---|
| | 18 | 19 | 20 | 21 | 22 | 23 | 24 | 25 | 26 | 27 | 28 |
| Polyethylene/ | 6 | 6 | 6 | 6 | 6 | 6 | 6 | 6 | 6 | 6 | 6 |
| microcrystalline wax | | | | | | | | | | | |
| Polysilicone-32 | 25 | 25 | 25 | 25 | 25 | 25 | 25 | 25 | 25 | 25 | 25 |
| Diphenylsiloxy phenyl | 29.98 | 29.98 | 29.98 | 29.98 | 29.98 | 29.98 | 29.98 | 29.98 | 29.98 | 29.98 | 29.98 |
| trimethicone | | | | | | | | | | | |
| Diphenyl dimethicone | — | — | — | — | — | — | — | — | — | — | — |
| PEG-10 dimethicone | 8 | — | — | — | — | — | — | — | — | — | — |
| PEG-12 dimethicone | — | 8 | — | — | — | — | — | — | — | — | — |
| PEG-10 methyl ether | — | — | 8 | — | — | — | — | — | — | — | — |
| dimethicone | | | | | | | | | | | |
| PEG-11 methyl ether | — | — | — | 8 | — | — | — | — | — | — | — |
| dimethicone | | | | | | | | | | | |
| PEG-9 | — | — | — | — | 8 | — | — | — | — | — | — |
| polydimethylsiloxyethyl | | | | | | | | | | | |
| dimethicone | | | | | | | | | | | |
| Lauryl PEG-9 | — | — | — | — | — | 8 | — | — | — | — | — |
| polydimethylsiloxyethyl | | | | | | | | | | | |
| dimethicone | | | | | | | | | | | |
| bis-Butyldimethicone | — | — | — | — | — | — | 8 | — | — | — | — |
| polyglyceryl-3 | | | | | | | | | | | |
| Cetyl PEG/PPG-10/1 | — | — | — | — | — | — | — | 8 | — | — | — |
| dimethicone | | | | | | | | | | | |
| (Dimethicone/(PEG- | — | — | — | — | — | — | — | — | 8 | — | — |
| 10/15)) crosspolymer⁽²⁾ | | | | | | | | | | | |
| (Dimethicone/ | — | — | — | — | — | — | — | — | — | 8 | — |
| polyglycerin-3) | | | | | | | | | | | |
| crosspolymer⁽³⁾ | | | | | | | | | | | |
| (PEG-15/lauryl | — | — | — | — | — | — | — | — | — | — | 8 |
| polydimethylsiloxyethyl | | | | | | | | | | | |
| dimethicone) | | | | | | | | | | | |
| crosspolymer⁽⁴⁾ | | | | | | | | | | | |
| Mica⁽¹⁾ | 15 | 15 | 15 | 15 | 15 | 15 | 15 | 15 | 15 | 15 | 15 |
| Polyglyceryl-2 | 10 | 10 | 10 | 10 | 10 | 10 | 10 | 10 | 10 | 10 | 10 |
| triisostearate | | | | | | | | | | | |
| Hydrogenated | 5 | 5 | 5 | 5 | 5 | 5 | 5 | 5 | 5 | 5 | 5 |
| polyisobutene | | | | | | | | | | | |
| Dimethicone | 0.02 | 0.02 | 0.02 | 0.02 | 0.02 | 0.02 | 0.02 | 0.02 | 0.02 | 0.02 | 0.02 |
| Red No. 202 | 1 | 1 | 1 | 1 | 1 | 1 | 1 | 1 | 1 | 1 | 1 |
| Total | 100 | 100 | 100 | 100 | 100 | 100 | 100 | 100 | 100 | 100 | 100 |
| Secondary adhesion-less | A | A | A | A | A | A | A | A | A | A | A |
| effects | | | | | | | | | | | |

⁽²⁾KSG-210 (Shin-Etsu Chemical Co., Ltd.)
⁽³⁾KSG-710 (Shin-Etsu Chemical Co., Ltd.)
⁽⁴⁾KSG-360Z (Shin-Etsu Chemical Co., Ltd.)

TABLE 2B

| | Ex. | Ex. | Ex. | Ex. | Ex. | Ex. | Ex. | Ex. | Ex. | Ex. | Ex. |
|---|---|---|---|---|---|---|---|---|---|---|---|
| | 29 | 30 | 31 | 32 | 33 | 34 | 35 | 36 | 37 | 38 | 39 |
| Polyethylene/ | 6 | 6 | 6 | 6 | 6 | 6 | 6 | 6 | 6 | 6 | 6 |
| microcrystalline wax | | | | | | | | | | | |
| Polysilicone-32 | — | — | — | — | — | — | — | — | — | — | — |
| Diphenylsiloxy phenyl | 29.98 | 29.98 | 29.98 | 29.98 | 29.98 | 29.98 | 29.98 | 29.98 | 29.98 | 29.98 | 29.98 |
| trimethicone | | | | | | | | | | | |
| Diphenyl dimethicone | 25 | 25 | 25 | 25 | 25 | 25 | 25 | 25 | 25 | 25 | 25 |
| PEG-10 dimethicone | 8 | — | — | — | — | — | — | — | — | — | — |
| PEG-12 dimethicone | — | 8 | — | — | — | — | — | — | — | — | — |
| PEG-10 methyl ether | — | — | 8 | — | — | — | — | — | — | — | — |
| dimethicone | | | | | | | | | | | |
| PEG-11 methyl ether | — | — | — | 8 | — | — | — | — | — | — | — |
| dimethicone | | | | | | | | | | | |
| PEG-9 | — | — | — | — | 8 | — | — | — | — | — | — |
| polydimethylsiloxyethyl | | | | | | | | | | | |
| dimethicone | | | | | | | | | | | |
| Lauryl PEG-9 | — | — | — | — | — | 8 | — | — | — | — | — |
| polydimethylsiloxyethyl | | | | | | | | | | | |
| dimethicone | | | | | | | | | | | |
| bis-Butyldimethicone | — | — | — | — | — | — | 8 | — | — | — | — |
| polyglyceryl-3 | | | | | | | | | | | |
| Cetyl PEG/PPG-10/1 | — | — | — | — | — | — | — | 8 | — | — | — |
| dimethicone | | | | | | | | | | | |
| (Dimethicone/(PEG- | — | — | — | — | — | — | — | — | 8 | — | — |
| 10/15)) crosspolymer⁽²⁾ | | | | | | | | | | | |
| (Dimethicone/ | — | — | — | — | — | — | — | — | — | 8 | — |
| polyglycerin-3) | | | | | | | | | | | |
| crosspolymer⁽³⁾ | | | | | | | | | | | |
| (PEG-15/lauryl | — | — | — | — | — | — | — | — | — | — | 8 |
| polydimethylsiloxyethyl | | | | | | | | | | | |
| dimethicone) | | | | | | | | | | | |
| crosspolymer⁽⁴⁾ | | | | | | | | | | | |
| Mica⁽¹⁾ | 15 | 15 | 15 | 15 | 15 | 15 | 15 | 15 | 15 | 15 | 15 |
| Polyglyceryl-2 | 10 | 10 | 10 | 10 | 10 | 10 | 10 | 10 | 10 | 10 | 10 |
| triisostearate | | | | | | | | | | | |
| Hydrogenated | 5 | 5 | 5 | 5 | 5 | 5 | 5 | 5 | 5 | 5 | 5 |
| polyisobutene | | | | | | | | | | | |
| Dimethicone | 0.02 | 0.02 | 0.02 | 0.02 | 0.02 | 0.02 | 0.02 | 0.02 | 0.02 | 0.02 | 0.02 |
| Red No. 202 | 1 | 1 | 1 | 1 | 1 | 1 | 1 | 1 | 1 | 1 | 1 |
| Total | 100 | 100 | 100 | 100 | 100 | 100 | 100 | 100 | 100 | 100 | 100 |
| Secondary adhesion-less | A | A | A | A | A | A | A | A | A | A | A |
| effects | | | | | | | | | | | |

As indicated in Table 2A and Table 2B above, excellent secondary adhesion-less effects were obtained when using any of (i) a polyoxyalkyene-modified silicone, (ii) a polyoxyalkylene/alkyl-comodified silicone, (iii) a polyglycerin-modified silicone and (iv) a partially crosslinked-type silicone gel as the (B) silicone-based surfactant (Examples 18 to 39).

### Examples 40 to 56

Oil-based lip cosmetics having the compositions described in Table 3A and Table 3B below were prepared, and the secondary adhesion-less effects were evaluated.

TABLE 3A

| | Ex. | Ex. | Ex. | Ex. | Ex. | Ex. | Ex. | Ex. | Ex. |
|---|---|---|---|---|---|---|---|---|---|
| | 40 | 41 | 42 | 43 | 44 | 45 | 46 | 47 | 48 |
| Polyethylene/microcrystalline | 6 | 6 | 6 | 6 | 6 | 6 | 6 | 6 | 6 |
| wax | | | | | | | | | |
| Polysilicone-32 | 25 | 25 | 25 | 25 | 25 | 25 | 25 | 25 | 25 |
| Diphenylsiloxy phenyl | 29.98 | 29.98 | 29.98 | 29.98 | 29.98 | 29.98 | 29.98 | 29.98 | 29.98 |
| trimethicone | | | | | | | | | |
| PEG-10 dimethicone | 8 | 8 | 8 | 8 | 8 | 8 | 8 | 8 | 8 |
| Mica⁽⁵⁾ | 15 | — | — | — | — | — | — | — | — |
| Mica⁽⁶⁾ | — | 15 | — | — | — | — | — | — | — |
| Mica⁽⁷⁾ | — | — | 15 | — | — | — | — | — | — |
| Silica⁽⁸⁾ | — | — | — | 15 | — | — | — | — | — |
| Silica⁽⁹⁾ | — | — | — | — | 15 | — | — | — | — |
| Silica⁽¹⁰⁾ | — | — | — | — | — | 15 | — | — | — |
| Mica/silica⁽¹¹⁾ | — | — | — | — | — | — | 15 | — | — |
| Synthetic phlogopite⁽¹²⁾ | — | — | — | — | — | — | — | 15 | — |
| Synthetic phlogopite⁽¹³⁾ | — | — | — | — | — | — | — | — | 15 |
| Polyglyceryl-2 triisostearate | 10 | 10 | 10 | 10 | 10 | 10 | 10 | 10 | 10 |
| Hydrogenated polyisobutene | 5 | 5 | 5 | 5 | 5 | 5 | 5 | 5 | 5 |
| Dimethicone | 0.02 | 0.02 | 0.02 | 0.02 | 0.02 | 0.02 | 0.02 | 0.02 | 0.02 |
| Red No. 202 | 1 | 1 | 1 | 1 | 1 | 1 | 1 | 1 | 1 |
| Total | 100 | 100 | 100 | 100 | 100 | 100 | 100 | 100 | 100 |
| Secondary adhesion-less | B | A | B | A | B | B | A | A | B |
| effects | | | | | | | | | |

⁽⁵⁾Average particle size 7.7 μm
⁽⁶⁾Average particle size 5.2 μm
⁽⁷⁾Average particle size 5.0 μm
⁽⁸⁾Average particle size 7.9 μm
⁽⁹⁾Average particle size 2-4 μm
⁽¹⁰⁾Average particle size 4.5-6 μm
⁽¹¹⁾Complex powder with average particle size 10-50 μm
⁽¹²⁾Average particle size 2.4-2.9 μm
⁽¹³⁾Average particle size 4.6 μm

TABLE 3B

| | Ex. | Ex. | Ex. | Ex. | Ex. | Ex. | Ex. | Ex. |
|---|---|---|---|---|---|---|---|---|
| | 49 | 50 | 51 | 52 | 53 | 54 | 55 | 56 |
| Polyethylene/microcrystalline wax | 6 | 6 | 6 | 6 | 6 | 6 | 6 | 6 |
| Polysilicone-32 | 25 | 25 | 25 | 25 | 25 | 25 | 25 | 25 |
| Diphenylsiloxy phenyl trimethicone | 29.98 | 29.98 | 29.98 | 29.98 | 29.98 | 29.98 | 29.98 | 29.98 |
| PEG-10 dimethicone | 8 | 8 | 8 | 8 | 8 | 8 | 8 | 8 |
| Synthetic phlogopite⁽¹⁴⁾ | 15 | — | — | — | — | — | — | — |
| Synthetic phlogopite⁽¹⁵⁾ | — | 15 | — | — | — | — | — | — |
| Boron nitride⁽¹⁶⁾ | — | — | 15 | — | — | — | — | — |
| Barium sulfate⁽¹⁷⁾ | — | — | — | 15 | — | — | — | — |
| (Vinyl dimethicone/methicone | — | — | — | — | 15 | — | — | — |
| silsesquioxane) crosspolymer⁽¹⁸⁾ | | | | | | | | |
| (Diphenyl dimethicone/vinyl | — | — | — | — | — | 15 | — | — |
| diphenyl dimethicone/ | | | | | | | | |
| silsesquioxane) crosspolymer⁽¹⁹⁾ | | | | | | | | |
| (HDI/trimethylol hexyl lactone) | — | — | — | — | — | — | 15 | — |
| crosspolymer⁽²⁰⁾ | | | | | | | | |
| Cellulose⁽²¹⁾ | — | — | — | — | — | — | — | 15 |
| Polyglyceryl-2 triisostearate | 10 | 10 | 10 | 10 | 10 | 10 | 10 | 10 |
| Hydrogenated polyisobutene | 5 | 5 | 5 | 5 | 5 | 5 | 5 | 5 |
| Dimethicone | 0.02 | 0.02 | 0.02 | 0.02 | 0.02 | 0.02 | 0.02 | 0.02 |
| Red No. 202 | 1 | 1 | 1 | 1 | 1 | 1 | 1 | 1 |
| Total | 100 | 100 | 100 | 100 | 100 | 100 | 100 | 100 |
| Secondary adhesion-less effects | A | A | B | A | B | A | A | A |

⁽¹⁴⁾Average particle size 5-7 μm
⁽¹⁵⁾Average particle size 14.3 μm
⁽¹⁶⁾Average particle size 5.8 μm
⁽¹⁷⁾Average particle size 5 μm
⁽¹⁸⁾Average particle size 5 μm
⁽¹⁹⁾Average particle size 5 μm
⁽²⁰⁾Average particle size 10-15 μm
⁽²¹⁾Average particle size 5-25 μm

As indicated in Table 3A and Table 3B above, excellent secondary adhesion-less effects were obtained when using either of (i) an extender pigment and (ii) a polymer powder as the (C) powder component (Examples 40 to 56).

### Comparative Examples 3 to 6

The oil-based lip cosmetics having the compositions described in Table 4 below were prepared and evaluated regarding secondary adhesion-less effects. In order to facilitate comparison, Examples 1, 10 and 11 are also shown.

TABLE 4

| | | | | Comp. | Comp. | Comp. | Comp. |
|---|---|---|---|---|---|---|---|
| | Ex. 1 | Ex. 10 | Ex. 11 | Ex. 3 | Ex. 4 | Ex. 5 | Ex. 6 |
| Polyethylene/microcrystalline wax | 6 | 6 | 6 | 6 | 6 | 6 | 6 |
| Polysilicone-32 | 25 | 25 | 25 | 25 | 25 | 25 | 25 |
| Diphenylsiloxy phenyl trimethicone | 29.98 | 34.98 | 39.98 | 42.98 | 39.98 | 38.98 | 34.98 |
| PEG-10 dimethicone | 8 | 8 | 8 | 8 | 8 | 8 | 8 |
| Mica⁽¹⁾ | 15 | 10 | 5 | — | — | — | — |
| Polyglyceryl-2 triisostearate | 10 | 10 | 10 | 10 | 10 | 10 | 10 |
| Hydrogenated polyisobutene | 5 | 5 | 5 | 5 | 5 | 5 | 5 |
| Dimethicone | 0.02 | 0.02 | 0.02 | 0.02 | 0.02 | 0.02 | 0.02 |
| Pearlescent pigment | 0 | 0 | 0 | 2 | 5 | 6 | 10 |
| Red No. 202 | 1 | 1 | 1 | 1 | 1 | 1 | 1 |
| Total | 100 | 100 | 100 | 100 | 100 | 100 | 100 |
| Secondary adhesion-less effects | A | A | A | C | C | C | C |

As indicated in Table 4 above, when a pearlescent pigment, which is a powder not corresponding to the (C) powder component, was blended instead of mica, adequate secondary adhesion-less effects were not obtained (Comparative Examples 3 to 6).

Hereinafter, an example of a formulation for the oil-based lip cosmetic of the present invention will be indicated. The present invention is not limited in any way by the formulation example below and, needless to say, is defined by the claims. The blended amounts are indicated in percentage by mass with respect to the overall amount of the oil-based lip cosmetic.

### Formulation Example 1: Oil-Based Lip Cosmetic

| (Component name) | Blended amount (mass %) |
|---|---|
| Polyethylene wax | 5.3 |
| Microcrystalline wax | 0.9 |
| Diphenylsiloxy phenyl trimethicone | 29.18 |
| Diphenyl dimethicone | 25 |
| PEG-10 dimethicone | 8 |
| Mica | 15 |
| Polyglyceryl-2 triisostearate | 10 |
| Hydrogenated polyisobutene | 5 |
| Dimethicone | 0.02 |
| Red No. 202 | 1 |
| (Li/Mg/Na) silicate | 0.1 |
| Argania spinosa kernel oil | 0.1 |
| Glucosyl hesperidin | 0.1 |
| Allantoin | 0.1 |
| Glycerin | 0.1 |
| Ascorbic acid | 0.1 |

1. An oil-based lip cosmetic comprising:
(A) a phenyl-modified silicone;
(B) 1.5% to 15% by mass, with respect to the overall amount of the cosmetic, of a silicone-based surfactant; and
(C) 5% to 35% by mass, with respect to the overall amount of the cosmetic, of a powder component selected from among (i) extender pigments and (ii) polymer powders.

2. The oil-based lip cosmetic according to claim 1, wherein the (A) phenyl-modified silicone comprises diphenyl dimethicone.

3. The oil-based lip cosmetic according to claim 1, wherein the (B) silicone-based surfactant is selected from among (i) polyoxyalkylene-modified silicones, (ii) polyoxyalkylene/alkyl-comodified silicones, (iii) polyglycerin-modified silicones, and (iv) partially-crosslinked-type modified silicone gels.

4. The oil-based lip cosmetic according to claim 1, wherein the (B) silicone-based surfactant comprises PEG-10 dimethicone or PEG-12 dimethicone.

5. The oil-based lip cosmetic according to claim 1, wherein the average particle size of the (C) powder component is 2 to 20 μm.

6. The oil-based lip cosmetic according to claim 1, wherein the (C) (i) extender pigments are of one or more types selected from the group consisting of mica, silica, alumina, barium sulfate, titanium oxide, synthetic phlogopite, boron nitride, and complex powders thereof.

7. The oil-based lip cosmetic according to claim 1, wherein the (C) (ii) polymer powders are of one or more types selected from the group consisting of (vinyl dimethicone/methicone silsesquioxane) crosspolymer, (diphenyl dimethicone/vinyl diphenyl dimethicone/silsesquioxane) crosspolymer, cellulose and (HDI/trimethylol hexyl lactone) crosspolymer.

8. The oil-based lip cosmetic according to claim 1, further comprising an oil-based gelling agent.

9. The oil-based lip cosmetic according to claim 1, wherein the oil-based lip cosmetic has a water content of 2% by mass or less.
